(19) United States
(12) Patent Application Publication
(10) Pub. No.: US 2026/0232224 A1
(43) Pub. Date: Aug. 13, 2026

(54) WEARABLE DEVICE TO MONITOR MEDICAL STATE

(71) Applicant: Boomboxdr LLC, Madison, MS (US)

(72) Inventor: Donald Glidewell, Madison, MS (US)

(21) Appl. No.: 19/538,436

(22) Filed: Feb. 12, 2026

Related U.S. Application Data
(60) Provisional application No. 63/758,192, filed on Feb. 13, 2025.

(51) Int. Cl. A61B 5/11; A61B 5/00

(52) U.S. Cl. CPC A61B 5/112; A61B 5/0002; A61B 5/6807; A61B 5/7267; A61B 5/7275; A61B 5/742; A61B 5/748; A61B 2560/0214

(57) ABSTRACT

A system can include a device and one or more computing devices. The device can be disposed within a wearable article. The device can include one or more processing circuits. The one or more processing circuits can receive medical data associated with a user of the device and measured by one or more sensors of the device, transmit one or more first data packets to provide the medical data to the one or more computing devices. The one or more computing devices can receive the one or more first data packets, retrieve a data structure associated with the user, update the data structure to reflect at least a portion of the medical data, and transmit one or more second data packets to indicate receipt of the medical data.

### CROSS-REFERENCE TO RELATED PATENT APPLICATION

This application claims the benefit of and priority to U.S. Provisional Patent Application No. 63/758,192, filed Feb. 13, 2025, the entirety of which is incorporated by reference herein.

### BACKGROUND

Long-term care (“LTC”) facilities provide services for residents.

### SUMMARY

At least one embodiment relates to a device. The device can be disposed within a wearable article of clothing. The device can include one or more sensors. The one or more sensors can collect information associated with a user of the device. The device can also include a communication interface. The communication interface can communicate with one or more computing devices. The device can also include a power source. The power source can power the one or more sensors, the communication interface, and one or more processing circuits of the device. The one or more processing circuits can be in communication with the one or more sensors and the communication interface. The one or more processing circuits can receive, from the one or more sensors, the information associated with the user of the device. The information can include medical data measured by the one or more sensors. The one or more processing circuits can also transmit, via the communication interface responsive to receipt of the medical data measured by the one or more sensors, one or more signals to provide the medical data measured by the one or more sensors to the one or more computing devices. The one or more processing circuits can also receive, from the one or more computing devices responsive to transmission of the one or more signals, a response to indicate receipt of the medical data measured by the one or more sensors.

At least one embodiment relates to a system. The system can include one or more memory devices. The one or more memory devices can store instructions thereon. The instructions can cause, when executed by one or more processors, the one or more processors to receive, from a device configured to be disposed within a wearable article of clothing, medical data measured by one or more sensors of the device. The instructions can also cause the one or more processors to detect, responsive to receipt of the medical data measured by the one or more sensors of the device, a difference between one or more portions of the medical data measured by the one or more sensors of the device and one or more portions of previous medical data measured by the one or more sensors of the device. The instructions can also cause the one or more processors to input, responsive to detection of the difference, at least the one or more portions of the medical data measured by the one or more sensors into a Machine Learning (ML) model trained to predict medical statuses. The instructions can also cause the one or more processors to generate, using the ML model, a prediction of a medical status or medical state of a user of the device. The prediction can be based on an output of the ML model. The instructions can also cause the one or more processors to transmit, responsive to generation of the prediction, one or more signals to cause a display device to display a user interface to provide an indication of the prediction of the medical status of the user of the device.

At least one embodiment relates to a system. The system can include a device. The device can be disposed within a wearable article. The device can include one or more sensors. The one or more sensors can collect information associated with a user of the device. The device can include a communication interface. The communication interface can communicate with one or more computing devices of the system. The one or more computing devices can be remote from the device. The device can include a power source. The power source can power the one or more sensors, the communication interface, and one or more processing circuits of the device. The one or more processing circuits can communicate with the one or more sensors and the communication interface. The one or more processing circuits can receive, from the one or more sensors, the information associated with the user of the device. The information can include medical data of the user. The medical data can be measured by the one or more sensors. The one or more processing circuits can transmit, via the communication interface, responsive to receipt of the medical data, one or more first data packets to provide the medical data to the one or more computing devices. The one or more computing devices can receive, from the communication interface, the one or more first data packets. The one or more computing devices can retrieve, from at least one database, a data structure associated with the user. The one or more computing devices can update, responsive to retrieval of the data structure, the data structure to reflect at least a portion of the medical data. The one or more computing devices can transmit, to the communication interface, one or more second data packets to indicate receipt of the medical data.

At least one embodiment relates to a distributed computing system. The distributed computing system can include a device. The device can be disposed within a wearable article. The device can include one or more processing circuits. The one or more processing circuits can receive, from one or more sensors of the device, information associated with a user of the device. The information can include medical data of the user. The medical data can be measured by the one or more sensors. The one or more processing circuits can transmit, via a communication interface, responsive to receipt of the medical data, one or more first data packets to provide the medical data to one or more computing devices of the distributed computing system. The one or more computing devices can receive, from the communication interface, the one or more first data packets. The one or more computing devices can retrieve, from at least one database, a data structure associated with the user. The one or more computing devices can update, responsive to retrieval of the data structure, the data structure to reflect at least a portion of the medical data. The one or more computing devices can transmit, to the communication interface, one or more second data packets to indicate receipt of the medical data measured by the one or more sensors.

At least one embodiment relates to a device. The device can be disposed within a wearable article. The device can include one or more sensors. The one or more sensors can collect information associated with a user of the device. The device can include a communication interface. The communication interface can communicate with one or more computing devices. The one or more computing devices can be remote from the device. The device can include a power source. The power source can power the one or more sensors, the communication interface, and one or more processing circuits of the device. The one or more processing circuits can communicate with the one or more sensors and the communication interface. The one or more processing circuits can receive, from the one or more sensors, the information associated with the user of the device. The information can include medical data of the user. The medical data can be measured by the one or more sensors. The one or more processing circuits can transmit, via the communication interface, responsive to receipt of the medical data, one or more first data packets to provide the medical data to the one or more computing devices. The one or more processing circuits can receive, from the one or more computing devices, one or more second data packets to indicate an update to a data structure associated with the user.

Any discussion within this summary is illustrative only and should not be regarded as being limiting in any way.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram of a system to monitor medical statuses of one or more residents of a facility, in accordance with an embodiment.

FIG. 2 is a sequence diagram illustrating communication between components of the system illustrated in FIG. 1, in accordance with an embodiment.

FIG. 3 is a flow diagram of a process to monitor medical statuses, in accordance with an embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, systems and methods for resident monitoring are described herein. Resident monitoring may include collecting medical data and analyzing the medical data to detect changes. Resident monitoring may include utilizing wearable devices that collect information associated with one or more residents. For example, a resident of a LTC facility may wear an article of clothing that includes one or more sensors. To continue this example, the sensors may provide data to a computing device. The computing device may be a local device or a remote device (e.g., servers, remote databases, etc.). Resident monitoring may provide for proactive measures to be taken. For example, some residents may struggle or lack an ability to communicate which makes providing care difficult. The implementation of resident monitoring with wearable devices can assist in caring for residents that struggle to communicate.

Other systems and/or devices may provide devices to collect medical data, however these systems are often obtrusive which often leads to residents being unwilling to utilize these devices. For example, a device that is placed on a resident's finger may limit or reduce the resident's use of their hands. To continue this example, devices that are placed on fingers are prone to falling off or disengaging with residents. As a result, consistent and accurate data collection is reduced as there is often gaps or voids in the collection of the data. Furthermore, the noticeability of these devices may impact a wellbeing of the residents as they may feel uncomfortable or nervous with these devices.

Some technical solutions of the present disclosure include a device that can be disposed within an article of clothing. The placement of the device within articles of clothing reduces or eliminates noticeability of the device. The concealment of the device provides residents with assurances that the device will not be easily noticed. The device may include sensors or data collection devices. The sensors may measure, obtain, or otherwise collect information associated with the residents. For example, the sensors may measure a heart rate of the residents. As another example, the sensors may measure or detect motion.

Moreover, the device may communicate with one or more computing devices. For example, the device may include a communication interface to communicate with a server bank. The device may provide (to the computing devices) collected medical data and the computing devices may process, analyze, or otherwise evaluate the medical data. The computing devices may monitor residents of one or more LTC facilities based on the medical data collected by the device. For example, the computing devices may monitor changes in position of a resident. As another example, the computing device may monitor changes in heart rate of a resident.

In some embodiments, the device may be provided with one or more articles of clothing. For example, the device may be included with a sock. As another example, the device may be added to or placed within the article of clothing. The device may collect medical data to assist in providing care for residents having one or more medical conditions. For example, the medical data collected by the device may assist in caring for residents having dementia, autism, communication difficulties, prone to falling, movement reduction, and among other medical conditions.

In some embodiments, the sensors may collect information to track medical information such as heart rate, sweat levels and/or amounts of perspiration, motion, anxiety levels, and among other medical information. For example, the sensors may include galvanic skin response (GSR) sensors, accelerometers, motion sensors, and/or optical heart rate sensors. In some embodiments, the article or articles of clothing may include textile material that can incorporate or otherwise house the sensors. For example, the articles of clothing may include slots or pouches that can receive or house the sensors. The slots may be positioned within an interior portion or area of the articles of clothing such that the sensors may interface with or otherwise come into contact with the skin of the user. In some embodiments, the articles of clothing may include material similar to normal clothing. For example, the sensors may be located in socks and the socks may include material such as cotton, polyester, wool, and/or other possible materials.

In some embodiments, the device may communicate with remote databases to implement and/or utilize one or more Machine Learning (ML) models. For example, the device may provide medical data to a remote processing system and the remote processing system may generate one or more predictions, such as predictions regarding a medical state or a medical status of a user of the device. In some embodiments, the processing systems may utilize ML models to detect statuses of one or more residents. For example, ML models may be used to detect signs of stress, agitation, irritability, anxiety, and/or discomfort. In some embodiments, the device may store, utilize, and/or access one or more of the ML models described herein.

Advantageously, the medical data collected by the device may assist in detecting status changes or state changes in residents that have difficulty communicating. For example, a resident may be unable to and/or struggle to communicate how they are feeling, and the medical data collected by the device may assist a care giver in knowing a status of the resident.

In some embodiments, the device may include at least one communication interface to communicate with one or more remote devices. For example, the device may include a communication interface to communicate via Bluetooth. As another example, the device may include a communication interface to communicate via a Wide Area Network (WAN).

In some embodiments, the device may include one or more power sources. For example, the device may include batteries. In some embodiments, the power sources may power and/or provide power to one or more components of the device. For example, the power sources may power the sensors. In some embodiments, the power sources may include one or more types of energy storage devices. For example, the power sources may include lithium batteries, alkaline batteries, rechargeable batteries, disposable batteries, and/or other possible energy storage devices.

As a non-limiting example, the sensors may collect medical data that includes heart rate, sweat levels, and movement data or movement patterns. In this non-limiting example, the medical data may correspond to a resident that is wearing an article of clothing (e.g., a sock, an elastic bandage, a sleeve, etc.) that includes the device. To continue this non-limiting example, the medical data collected by the sensors may be provided to a processing system (e.g., servers, circuits, hardware, firmware, software, processing devices, etc.). In this non-limiting example, the processing system may utilize the medical data to detect that the resident is stressed. To continue this non-limiting example, changes in heart rate, sweat levels, or movement may indicate changes in a status of the resident.

In some embodiments, the device and/or one or more components thereof may be machine washable. For example, the device may be waterproof and as such an article of clothing that includes the device may be washed via a washing machine.

In some embodiments, the device may enhance or improve quality of life for the residents as the medical data collected by the device may assist caregivers in providing services to the residents. For example, medical data provided by the device may be used to detect changes in statuses of the residents. To continue this example, the changes in statuses may trigger or initiate prompts for the caregivers. In some embodiments, the device may provide medical data that assists in generating actionable insights. For example, the medical data may provide information to detect when a resident is feeling anxious. To continue this example, caregivers may be prompted and/or informed of the change in the status of the resident (e.g., the resident is feeling anxious).

In some embodiments, the device may collect information to detect changes over time. For example, the sensors may collect information that indicates movement patterns, balance, gait, weight distribution, and/or other possible movement metrics. In some embodiments, changes in movement patterns may be indicators of one or more potential medical conditions. For example, a change, a difference, a variation, or a deviation in a gait pattern (of a resident) may be a precursor or an indicator of multiple sclerosis, Parkinson's disease, or other medical conditions. In some embodiments, the changes over time may also provide indications of treatment efficacy. For example, the medical data collected by the device may provide indications that a resident's movement patterns has improved. In some embodiments, the medical data collected by the device may also assist in rehabilitation or physical therapy. For example, medical data that indicates gait patterns, movement patterns, and/or weight distribution may assist in monitoring progress.

In some embodiments, the device may collect information to assist in diabetic care for residents. For example, the sensors may collect information to detect or determine pressure points, reduced circulation, and/or other factors that may lead to complications. In some embodiments, the device may collect information to assist in monitoring or detecting resident falls. For example, the device may provide information that is used to detect or monitor changes in position. To continue this example, a constant position or minimal change in position for a given amount of time may indicate that a resident has fallen. In some embodiments, the device may collect information that indicates when residents are having difficulty walking and/or navigating.

In some embodiments, the devices may be utilized and/or worn by caregivers of the LTC facilities. For example, the device may be located in a sock and a caregiver may wear the sock. Stated otherwise, the sock may include one or more slots or receptacles for which the device and/or one or more components of the devices may be disposed or otherwise placed. In some embodiments, the device may collect information to assist in monitoring ergonomics for the caregivers. For example, the device may collect information to detect stress or strain that may be placed on the caregiver's feet. In some embodiments, the device may collect information to assist in reducing or limiting work-related injuries.

In some embodiments, the sensors may include at least one pressure sensors, accelerometers, gyroscopes, galvanic skin response (GSR) sensors, temperature sensors, heart rate sensors, flex sensors. The pressure sensors may collect, measure, and/or obtain information to monitor gait patterns, balance, weight distribution, or pressure points. The information collected by the pressure sensors may assist in one or more applications. For example, the information collected by the pressure sensors may assist in rehabilitation, physical therapy, diabetic foot care, movement detection, and/or fall detection. The accelerometers and/or the gyroscopes may collect, measure, and/or obtain information to track movement patterns, detect falls, or monitor medical conditions such as Parkinson's disease and/or multiple sclerosis. The information collected by the accelerometers and/or the gyroscopes may assist in rehabilitation, sports performance, and/or occupational health. The GSR sensors may collect, measure, and/or obtain information to measure sweat levels. The information collected by the GSR sensors may include indicators of stress or agitation, which may assist in detecting distress. The temperature sensors may collect, measure, and/or obtain information to monitor diabetic foot care such as ulcers or infections. The heart rate sensors may collect, measure, and/or obtain information to indicate stress levels, cardiovascular health, and/or to monitor neurological disorders. The flex sensors may collect, measure, and/or obtain information to monitor movement and/or exercises.

In some embodiments, the device may transmit the medical data via one or more connection modules, such as network jacks, communication devices, network switches, among other possibilities. For example, the device may include a Bluetooth module to transmit data via Bluetooth. In some embodiments, the device may transmit data to a smartphone (e.g., a user device) and/or a monitoring system (e.g., remote database, processing system, computing device, computing system, etc.). The data transmitted by the device may input into a ML model and the outputs, of the ML model, may assist in determining and/or detecting statuses of the residents.

In some embodiments, the ML models described herein may include one or more ML models trained for given applications. For example, the ML models may include a first model trained for dementia care applications. As another example, the ML models may include a second model trained for physical therapy applications. In some embodiments, the ML models may be continuously and/or semi-continuously updated and/or retained based on subsequent data or datasets. In some embodiments, the ML models may undergo training and validation. For example, predictions generated by the ML models may be compared with clinical diagnoses or medical testing. As another example, the ML models may be user tested. In some embodiments, the device may comply and/or conform with one more regulations. For example, the device may comply with healthcare guidelines that pertain to medical data storage and/or medical data transmission. The device may include memory or communicate with storage devices to house and/or maintain the medical data for subsequent utilization.

In some embodiments, the device may assist in monitoring sleep patterns. For example, the sensors can collect data to track movements during sleep, such as tossing and turning, which might indicate restlessness or sleep disturbances. As another example, the sensors may collect data to monitor changes in heart rate during sleep which may be indicative of sleep quality and/or may identify different sleep stages (like REM and deep sleep). As even another example, the sensors may include pulse oximeters to collect data to measure blood, which may assist in detecting conditions like sleep apnea.

In some embodiments, the device may assist in detecting neuropathy. For example, the sensors can collect information to detect changes in temperature patterns as these changes may be indicative of neuropathy. As another example, the sensors can collect information to detect changes in pressure distribution while walking, such as altered gait or foot mechanics which may be due to neuropathy. As even another example, the sensors may include bioimpedance sensors that can assess changes in tissue composition.

In some embodiments, the sensors may provide comprehensive real-time health data which may assist in early detection of distress or health issues. Early detection of distress or health issue may allow for prompt and/or effective intervention. In some embodiments, the monitoring of the information collected by the sensors may assist in generating care plans based on individual needs, which may improve the personalization and effectiveness of care. In some embodiments, burdens placed on caregivers may be reduced as the device may perform automated tasks to monitor statuses of residents. In some embodiments, the monitoring of statuses of the residents may provide comfort and/or peace of mind to family members and/or loved ones. For example, reports may be generated and/or provided to loved ones with information that indicates a status of a given resident. In some embodiments, the medical data collected by the device may assist in generating and/or providing training data for use in subsequent research or medical studies.

In some embodiments, the device may collect information to assist in detecting and/or treating at least one of Alzheimer's, fall risks, lymphedema, depression, renal failure, arthritis, COPD, Congestive heart failure, pneumonia, pressure ulcers, hypertension, diabetes, sepsis, anxiety, Parkinson's, and/or CAD. In some embodiments, the processing systems described herein may scrub and/or anonymize the medical data provided by the device and the processing system may utilize the anonymized data to assist in model training and/or model generation.

In some embodiments, the implementation of ML models to analyze and generate predictions (using the medical data collected by the device) may assist in preventive diagnoses and/or interventions. For example, the ML models may assist in early detection of dementia and Alzheimer's. To continue this example, ML models can be trained to analyze medical data to detect subtle changes in cognitive function, speech patterns, and daily activities recorded in medical notes, which may potentially indicate early signs of dementia or Alzheimer's. As another example, ML models may assist in fall risk assessment as ML models can be trained to detect residents that may be prone and/or likely to suffer a fall. To continue this example, the ML models may use information such as past incidents, mobility status, medication side effect, and/or other health conditions to generate fall risk predictions.

As even another example, ML models may assist in chronic disease management (e.g., diabetes, heart disease, arthritis, etc.) by identifying patterns in vitals, lab results, and symptoms, which may allow for timely interventions. As another example, ML models may assist with nutritional need analysis by analyzing dietary preferences, weight changes, blood tests, and other health indicators, which may lead to personalized nutrition plans.

As even another example, ML models may assist in medical optimization as ML models may predict adverse drug reactions or interactions in patients who often have complex medication regimens, which may improve medication safety. As another example, ML models may assist in predicting acute medical events as ML model can identify early signs of acute events such as strokes, heart attacks, or infections by analyzing changes in vital signs, lab results, and patient-reported symptoms.

As even another example, ML models may assist in monitoring mental health and/or well-being as ML models may identify signs of depression, anxiety, or social withdrawal, which often may go underdiagnosed in residents of LTC facilities. As another example, ML models may assist in mobility and/or rehabilitation as ML models may analyze physical therapy notes, mobility assessments, and daily activity logs to predict and optimize rehabilitation and mobility support needs.

As even another example, ML models may assist in preventive care recommendations as ML models can generate suggestions regarding preventive care measures such as vaccinations, screenings, or lifestyle modifications tailored to the elderly demographic. As another example, ML models may assist in identifying social and environmental factors as ML models may identify social determinants of health, such as isolation or lack of family support, which may play a role in holistic care at a LTC facility.

As a non-limiting example, the device described herein may also be implemented in, included with, and/or provided with a bed mat. In this non-limiting example, the bed mat may include and/or house the various sensors described herein. To continue this example, the sensors may collect information to monitor sleep patterns, including restlessness, sleep duration, and sleep cycles. In this non-limiting example, the device may communicate and/or transmit the information to the processing system and the processing system can detect and/or identify sleep disorders and/or sleep-related interventions. To continue this non-limiting example, the sensors may also collect information to monitor vital signs (e.g., heart rate, respiratory rate, blood pressure, etc.) and the processing system may use this information to detect changes in vital signs. In this non-limiting example, the sensors may also detect movement and the processing system may use this information to assess fall risks, physical assistance, or rehabilitation effectiveness. To continue this non-limiting example, the sensors may collect information to detect when a resident has gotten up from a bed and the processing system may generate prompts to assist in caring for residents that are prone to wonder. In this non-limiting example, the sensors may also collect information to monitor pressure points which may assist in preventing pressure ulcers (e.g., bedsores) by indicating points in time at which the resident may be moved or readjusted. To continue this non-limiting example, the sensors may also collect information to monitor environmental data such as temperature and/or humidity in a room. In this non-limiting example, the sensors may also collect information to detect moisture which may assist in caring for residents that suffer from incontinence and may also assist in maintaining hygiene and/or skin health. To continue this non-limiting example, the processing system may integrate and/or combine the information collected by the sensors with medical records and/or medical information. In this non-limiting example, the processing system may input and/or provide the integrated data into one or more ML models and the ML models may generate predictions based on the provided data. To continue this non-limiting example, the device may assist in providing behavioral health insights as changes in sleep patterns and/or activity levels may indicate depression, anxiety, and or cognitive decline and early detection may provide opportunities for early intervention.

### Article of Clothing With Device Integration

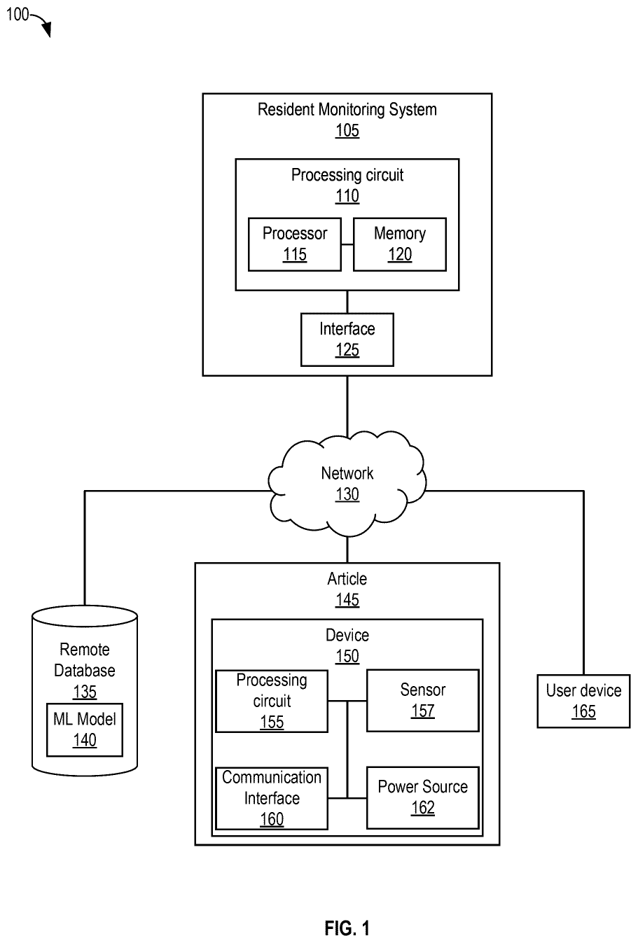

FIG. 1 depicts a block diagram of a system 100, according to some embodiments. Each system and/or component of the system 100 can include one or more processors, memory, network interfaces, communication interfaces, and/or user interfaces. Memory can store programming logic that, when executed by the processors, controls the operation of the corresponding computing system or device. Memory can also store data in databases. The network interfaces can allow the systems and/or components of the system 100 to communicate wirelessly. The communication interfaces can include wired and/or wireless communication interfaces and the systems and/or components of the system 100 can be connected via the communication interfaces. The various components in the system 100 can be implemented via hardware (e.g., circuitry), software (e.g., executable code), or any combination thereof. Systems, devices, and components in FIG. 1 can be added, deleted, integrated, separated, and/or rearranged.

In some embodiments, the system 100 may include at least one resident monitoring system (RMS) 105, at least one network 130, at least one remote database 135, at least one article 145, and at least one user device 165. In some embodiments, the system 100 and/or one or more systems, devices, and/or components thereof may implement at least one of the various techniques described herein to collect, monitor, or otherwise analyze medical states for one or more residents of a LTC facility.

In some embodiments, the RMS 105 may include at least one processing circuit 110 and at least one interface 125. The processing circuit 110 may include at least one processor 115 and memory 120. In some embodiments, the processing circuit 110 and/or one or more components thereof (e.g., the processors 115 and memory 120) may perform similar functionality to that of the RMS 105 and/or one or more components thereof. For example, memory 120 may store programming logic that, when executed by the processors 115, cause the processors 115 to perform functionality similar to that of the RMS 105. In some embodiments, the RMS 105 and/or one or more devices thereof may refer to or include one or more computing devices. For example, the RMS 105 may include a compute device that can provide one or more computing services or processing functions. As another example, the processing circuit 110 may be implemented within or otherwise included in one or more computing devices.

In some embodiments, the processing circuit 110 may be communicably connected to one or more components of the RMS 105. For example, the processing circuit 110 may be communicably connected to the interface 125. In some embodiments, the processors 115 may be implemented as a general-purpose processor, an application specific integrated circuit (ASIC), one or more field programmable gate arrays (FPGAs), a group of processing components, or other suitable electronic processing components.

In some embodiments, memory 120 (e.g., memory, memory unit, storage device, etc.) may include one or more devices (e.g., RAM, ROM, Flash memory, hard disk storage, etc.) for storing data and/or computer code for completing or facilitating the various processes, layers, and modules described in the present application. Memory 120 may be or include volatile memory or non-volatile memory. Memory 120 may include database components, object code components, script components, or any other type of information structure for supporting the various activities and information structures described in the present application. In some embodiments, memory 120 is communicably connected to the processors 115 via the processing circuit 110 and memory 120 includes computer code for executing (e.g., by the processing circuit 110 and/or the processors 115) one or more processes described herein.

In some embodiments, the interface 125 may include at least one of network communication devices, network interfaces, and/or other possible communication interfaces. The interface 125 may include wired or wireless communications interfaces (e.g., jacks, antennas, transmitters, receivers, transceivers, wire terminals, etc.) for conducting data communications with various systems, devices, and/or components described herein. The interface 125 may be direct (e.g., local wired or wireless communications) and/or via a communications network (e.g., the network 130). For example, the interface 125 may include an Ethernet card and port for sending and receiving data via an Ethernet-based communications link or network. As another example, the interface 125 may transmit, provide, or otherwise exchange data packets (with other devices) to exchange information or data. The interface 125 may also include a Wi-Fi transceiver for communicating via a wireless communications network (e.g., the network 130). The interface 125 may include a power line communications interface. The interface 125 may include an Ethernet interface, a USB interface, a serial communications interface, and/or a parallel communications interface. The interface 125 may include a Bluetooth interface or other devices for communicating via Bluetooth.

In some embodiments, the network 130 may include at least one of a local area network (LAN), wide area network (WAN), telephone network (such as the Public Switched Telephone Network (PSTN)), Controller Area Network (CAN), wireless link, intranet, the Internet, a cellular network, Bluetooth, and/or combinations thereof. In some embodiments, the various systems, components, and/or devices included in the system 100 may communicate with one another via the network 130.

In some embodiments, the remote database 135 may include at least one of a computing device, a remote server, a server bank, a remote device, and/or among other possible computer hardware and/or computer software. For example, the remote database 135 may include a server bank and the server bank can store, keep, maintain, and/or otherwise hold the various types of information described herein. In some embodiments, the remote database 135 may house and/or otherwise implement at least one of the various systems, devices, and/or components described herein. In some embodiments, the remote database 135 may include, store, maintain, and/or otherwise host the RMS 105. For example, the RMS 105 may be distributed across one or more servers (e.g., the remote database 135). In some implementations, the RMS 105 and/or various other components of the system 100 may be implemented using cloud computing services/platforms.

In some embodiments, the remote database 135 may house, store, maintain, or keep at least one machine learning models (shown as ML model 140). In some embodiments, the ML model 140 may refer to and/or include one or more ML models or artificial intelligence models. For example, the ML model 140 may include models trained using supervised learning. As another example, the ML model 140 may include a pre-trained model that was subsequently trained to generate at least one of the predictions described herein.

In some embodiments, the remote database 135 may house, store, maintain, or keep one or more data structures, data records, data objects, data arrays, database entries, among other possible forms of digital data. For example, the remote database 135 may store (as data structures) one or more sets of information (e.g., sensor measurements, medical data, etc.) provided by one or more devices described herein. As another example, the remote database 135 may maintain or otherwise keep an online data repository such that information or data (stored by the remote database 135) can be retrieved, accessed, or otherwise obtained from the remote database 135.

In some embodiments, the article 145 may refer to and/or include at least one of an article of clothing, a garment, a sock, an elastic bandage, a wrap, a compression sleeve, and/or other possible textile material or objects. The article 145 may include at least one device 150. For example, the device 150 may be disposed within a compartment of the article 145. In some embodiments, the device 150 may be provided with the article 145. For example, the device 150 may be provided when the article 145 is obtained. As another example, the device 150 may be provided subsequent to obtaining the article. In some embodiments, the article 145 may be worn, used, interfaced with, or otherwise used by one or more users. For example, the article 145 may include a sock and a user may wear (e.g., use) the sock. As another example, the article 145 may be placed on and/or rested on a user. In some embodiments, the device 150 may include the device described herein.

In some embodiments, the device 150 may include at least one processing circuit 155, at least one sensor 157, at least one communication interface 160, and at least one power source 162. In some embodiments, the processing circuit 155 may include components and/or circuitry similar to that of the processing circuit 110. In some embodiments, the communication interface 160 may include components and/or circuitry similar to that of the interface 125. In some embodiments, the sensors 157 may include at least one of the various sensors described herein. For example, the sensors 157 may include accelerometers. In some embodiments, the power source 162 may include at least one of the various power sources described herein. For example, the power source 162 may include batteries. In some embodiments, one or more components of the device 150 may be communicably coupled to one another. For example, the sensors 157 may be communicably coupled to the processing circuit 155.

In some embodiments, the sensors 157 may collect information associated with a user. For example, the sensors 157 may collect information associated with a user that is interfacing with the device 150. As another example, the sensors 157 may collect information associated with a user that is wearing the article 145. In some embodiments, the communication interface 160 may communicate with one or more computing devices. For example, the communication interface 160 may communicate with the RMS 105.

In some embodiments, the user device 165 may perform various actions and/or access various types of information. The information may be provided over the network 130. In some embodiments, the user device 165 may perform similar functionality to that of at least one system, device, and/or component of the system 100. For example, the user device 165 may perform similar operations to that of the RMS 105. In some embodiments, the user device 165 may include one or more applications to receive information, display information, and/or receive user interactions with content displayed by the user device 165.

In some embodiments, the user device 165 may include at least one of a screen, a monitor, a visual display device, a touchscreen display, a television, a video display, a liquid crystal display (LCD), a light emitting diode (LED) display, a mobile device, a kiosk, a digital terminal, a mobile computing device, a desktop computer, a smartphone, a tablet, a smart watch, a smart sensor, and/or any other device that can facilitate providing, receiving, displaying and/or otherwise interacting with content (e.g., webpages, mobile applications, etc.). For example, the user device 165 may include displays that include a resistive touchscreen that can receive user input via interactions (e.g., touches) with the touchscreen.

In some embodiments, the device 150 can be disposed within a wearable article of clothing. For example, the device 150 can be disposed within the article 145. As another example, the device 150 can be located within a portion of the article 145. In some embodiments, the processing circuit 155 may be in communication with the sensors 157 and the communication interface 160. For example, the processing circuit 155 may transmit one or more signals to the sensors 157.

In some embodiments, the processing circuit 155 may receive information associated with a user. For example, the processing circuit 155 may receive medical data associated with a user that is wearing the article 145. In some embodiments, the processing circuit 155 may receive information from the sensors 157. For example, the processing circuit 155 may receive, from the sensors 157, information that was collected, measured, and/or obtained by the sensors 157. In some embodiments, the processing circuit 155 may receive information that includes medical data. For example, the processing circuit 155 may receive medical data that was measured by the sensors 157.

In some embodiments, the processing circuit 155 may transmit one or more signals. For example, the processing circuit 155 may transmit signals via the communication interface 160. In some embodiment, the processing circuit 155 may transmit signals responsive to receipt of information. For example, the processing circuit 155 may transmit one or more signals responsive to receiving medical data from the sensors 157. In some embodiments, the processing circuits 155 may transmit signals to provide information. For example, the processing circuit 155 may transmit signals to provide the medical data measured by the sensors 157. In some embodiments, the processing circuits 155 may transmit the signals to one or more computing devices. For example, the processing circuits 155 may transmit the signals to the RMS 105. As another example, the processing circuits 155 may transmit the signals to the remote database 135.

In some embodiments, the processing circuits 155 may receive one or more signals. For example, the processing circuits 155 may receive one or more responses (e.g., data packets) to information transmitted by the communication interface 160. For example, the processing circuits 155 may receive (from the RMS 105) a response to indicate receipt of medical data. As another example, the processing circuit 155 may receive (from the remote database 135) a response to confirm receipt of transmitted medical data.

In some embodiments, the processing circuits 110 may receive medical data. For example, the processing circuits 110 may receive medical data from the device 150. As another example, the processing circuit 110 may receive or retrieve medical data from the remote database 135. In some embodiments, the processing circuits 110 may receive medical data that was measured, collected, and/or obtained by the sensors 157. For example, the processing circuits 110 may receive, via the interface 125, medical data that was transmitted by the communication interface 160.

In some embodiments, the processing circuits 110 may detect one or more differences. For example, the processing circuits 110 may detect differences between one or more sets of medical data. In some embodiments, the processing circuits 110 may detect differences between one or more portions of medical data. For example, the processing circuits 110 may detect differences between a first portion of medical data measured by the sensors 157 and a first portion of medical data previously provided to the processing circuits 110 (e.g., historical data, data stored in the remote database 135, etc.).

In some embodiments, the processing circuits 110 may input one or more portions of medical data into the ML model 140. For example, the processing circuits 110 may input the medical data as a data vector into the ML model 140. As another example, the processing circuit 110 may provide data or inputs to the ML model 140 via one or more prompts or calls to the ML model 140. In some embodiments, the processing circuits 110 may input the medical data responsive to detections of one or more differences. For example, the processing circuits 110 may input the medical data responsive to detecting differences between one or more portions of medical data. In some embodiments, the processing circuits 110 may input the medical data to predict one or more medical statuses. For example, the ML model 140 may be trained to predict at least one of the various medical statuses, medical conditions, and/or medical diagnoses described herein.

In some embodiments, the processing circuits 110 may generate one or more predictions or outputs. For example, the processing circuits 110 may utilize and/or implement the ML model 140 to generate one or more predictions (e.g., outputs). In some embodiments, the processing circuits 110 may use the ML model 140 to generate a prediction of a medical status. For example, the processing circuits 110 may use the ML model 140 to generate a prediction of a medical status of a user that is wearing the article 145.

In some embodiments, the processing circuit 110 may transmit one or more signals. For example, the processing circuit 110 may transmit one or more signals via the interface 125. In some embodiments, the processing circuit 110 may transmit one or more signals to cause a display device (e.g., the user device 165) to display one or more user interfaces, such as graphical user interface, digital depictions of information, or electronic renders of data or information. In some embodiments, the user interface may include and/or provide an indication of the prediction of the medical status.

In some embodiments, the display device may refer to and/or include at least one of the various described herein. For example, the display device may include the user device 165. In some embodiments, the processing circuits 110 may detect one or more changes in a condition. For example, the processing circuits 110 may detect a change in a condition of a user that is wearing the article 145. In some embodiments, the processing circuits 110 may detect the change based on one or more predictions by the ML model 140. For example, the processing circuits 110 may detect the change based on a prediction of a medical status of a user wearing the article 145.

In some embodiments, the processing circuit 110 may transmit one or more signals responsive to the detection of the change in the condition. For example, the processing circuit 110 may transmit one or more signals to the user device 165 responsive to detecting a change in a condition of a user wearing the article 145. In some embodiments, the processing circuit 110 may transmit the signals to cause one or more computing devices to produce an alert. For example, the processing circuit 110 may transmit signals to cause the user device 165 to produce an alert. In some embodiments, the alert may indicate the change in the condition. The alert may include at least one of an audible alert or audible output, a visual alert or visual output, and/or a haptic alert or haptic output.

In some embodiments, the processing circuit 110 may receive one or more electronic communications. For example, the processing circuit 110 may receive one or more data packets from the device 150. As another example, the processing circuit 110 may receive one or more wireless communication signals. In some embodiments, the processing circuit 110 may receive one or more sets of information or data via electronic communications. For example, the processing circuit 110 may receive (from the device 150) one or more sets of medical data that was measured, collected, or otherwise obtained by the sensors 157. As another example, the processing circuit 110 may retrieve or otherwise access one or more data structures stored by the remote database 135.

In some embodiments, the processing circuit 110 may update one or more sets of data. For example, the processing circuit 110 may update a data structure (associated with a user of the device 150) to reflect or otherwise include medical data, such as medical data that was measured by the sensors 157. As another example, the processing circuit 110 may update one or more data structures to include outputs that were generated by the ML model 140. In some embodiments, the processing circuit 110 may update one or more data structures to provide for a continuous or semi-continuous data synchronization or incremental updates to the medical data for a user. Stated otherwise, the processing circuit 110 may update the data structures to avoid or limit data stagnation.

In some embodiments, the processing circuit 110 may call, utilize, or otherwise implement the ML model 140 to generate one or more outputs. For example, the processing circuit 110 may transmit one or more call functions to retrieve or otherwise access the ML model 140. As another example, the processing circuit 110 may transmit at least one prompt to cause the ML Model 140 to implement or perform one or more actions. In some embodiments, the actions (performed by the ML model 140) may include data aggregation, data collection, data analysis, or data comparison.

In some embodiments, the processing circuit 110 may provide (via or in conjunction with one or more prompts) medical data to cause the ML model 140 to generate one or more outputs. The outputs may include (for example) a prediction or a detection of a variation, deviation, or difference in a gait pattern of a user. In this example, the ML model 140 may detect the difference based on a comparison of recent measurements, previously measured, or identified values or metrics (e.g., data collected by the sensors 157) with information stored or previously provided to the remote database 135. As another example, the outputs may include a detection of a decrease in average movement speed for a user of the device 150.

In some embodiments, the outputs (or underlying measurements or predictions) may be compared to thresholds or criteria. For example, the processing circuit 110 may compare one or more outputs (generated by the ML model 140) with variation thresholds (e.g., predetermined thresholds). Stated otherwise, the processing circuit 110 may compare differences between historical data and recent data to determine whether the differences are within predetermined bounds or values. Additionally, or alternatively, the processing circuit 110 may compare the differences with historical trends of the user. For example, the predetermined threshold may be an average value or metric (for the user and for a particular measurement, such as heart rate, movement patterns, etc.) during a given time frame, such as the past week, the past month, the past 3 months, the past 6 months, among other time periods or durations.

In some embodiments, the processing circuit 110 may store or otherwise sort the data as one or more entries. For example, the processing circuit 110 may generate a data array that includes one or more entries or slots to represent respective values or metrics for the medical data. As another example, the processing circuit 110 may index or otherwise mark respective portions of the medical data within data structures or data arrays. In some embodiments, the processing circuit 110 may index the medical data to assist in subsequent retrieval or utilization by the ML model 140. For example, the processing circuit 110 may index a portion of the medical data (that pertains to gait measurements) as corresponding to a given data array that includes one or more entries, which represent one or more of stride time, step time, cadence, among other parameters.

In some embodiments, the ML model 140 may extract or otherwise identify one or more values from data. For example, the sensors 157 may collect or measure gait parameters (such as stride time, step time, cadence, etc.). In this example, the ML model 140 may extract the gait parameters from the medical data. As another example, the sensors 157 may collect or measure joint range of motion (e.g., angular measurements). In this example, the ML model 140 may extract the angular measurements from the medical data. In some embodiments, the ML model 140 may extract values from recent medical data (e.g., data which may not have been indexed or otherwise processed by the processing circuit 110).

In some embodiments, the ML model 140 may compare or otherwise evaluate data with one or more sets of indexed data. For example, the ML model 140 may compare extracted values (from recent medical data) with one or more entries of a data array (that is stored or accessible from the remote database 135). The ML model 140 may compare the extracted values with the entries to identify or otherwise determine one or more variances, deviations, or differences between respective values. For example, the ML model 140 may compare a recent (e.g., one or more measurements within the past 15 minutes, 30 minutes, or 1 hour) stride length value with one or more indexed or historical values (e.g., one or more measurements within the past day, week, month, or year). The ML model 140 may identify a deviation, a variation, or a difference when one or more values (of the recent data) is outside of a bound or otherwise exceeds a threshold (relative to the historical data).

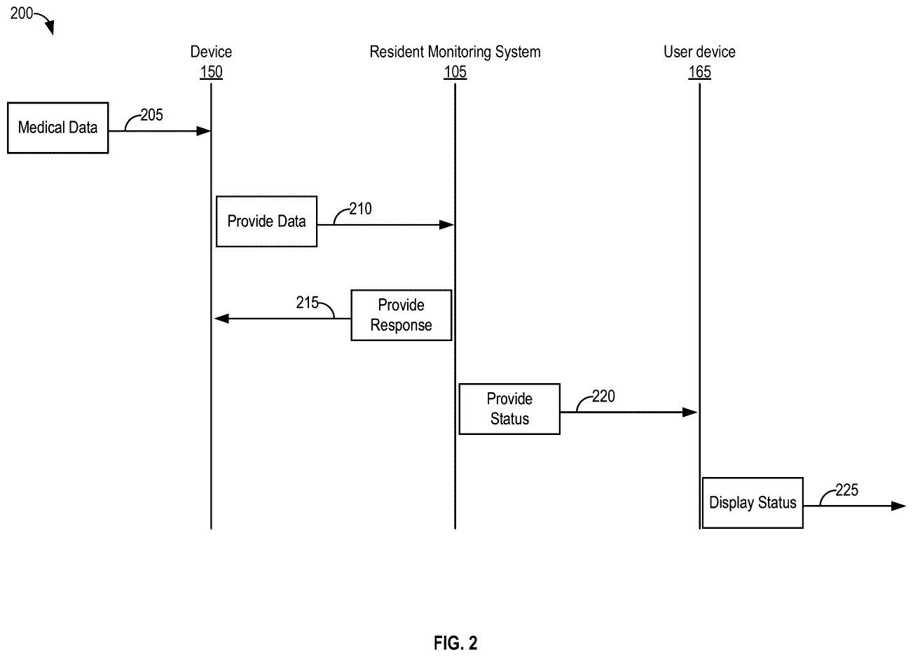

FIG. 2 depicts a sequence diagram of a process 200, according to some embodiments. In some embodiments, FIG. 2 may illustrate communication between one or more devices of the system 100. While the sequence diagram, as shown in FIG. 2, may illustrate communication in a given order or pattern, the illustration of FIG. 2 is in no way limiting. For example, a second step that may be shown in succession to (e.g., after, subsequent, following, etc.) a first step, may occur concurrently and/or simultaneously with the first step. As another example, one or more signals, as described with respect to FIG. 2, may be transmitted continuously and/or semi-continuously.

In some embodiments, step 205 may include receiving medical data. For example, the device 150 may receive medical data from the sensors 157. As another example, the sensors 157 may provide medical data to the processing circuits 155. In some embodiments, the device 150 may receive medical data, from the sensors 157, responsive to the sensors 157 measuring, collecting, and/or obtaining medical data associated with a user wearing the device 150.

In some embodiments, step 210 may include providing the medical data. For example, the processing circuits 155 may provide the medical data received in step 205. In some embodiments, the processing circuits 155 may provide the medical data to one or more devices. For example, the processing circuits 155 may provide the medical data to the RMS 105. As another example, the processing circuits 155 may provide the medical data to the remote database 135.

In some embodiments, step 215 may include providing one or more responses. For example, the RMS 105 may provide a response responsive to receiving the medical data in step 210. In some embodiments, the RMS 105 may provide the response to indicate receipt of the medical data. For example, the RMS 105 may provide the response to indicate, to the device 150, receipt of the medical data.

In some embodiments, step 220 may include providing one or more statuses. For example, the RMS 105 may provide one or more statuses (e.g., medical states, medical statuses, user condition, etc.) that were predicted by the ML model 140. In some embodiments, the RMS 105 may provide the statuses to the user device 165. Additionally, and/or alternatively, the RMS 105 may provide the statuses to the remote database 135.

In some embodiments, step 225 may include displaying one or more statuses. For example, the user device 165 may display the statuses provided in step 220. In some embodiments, the user device 165 may display the statuses via one or more display devices.

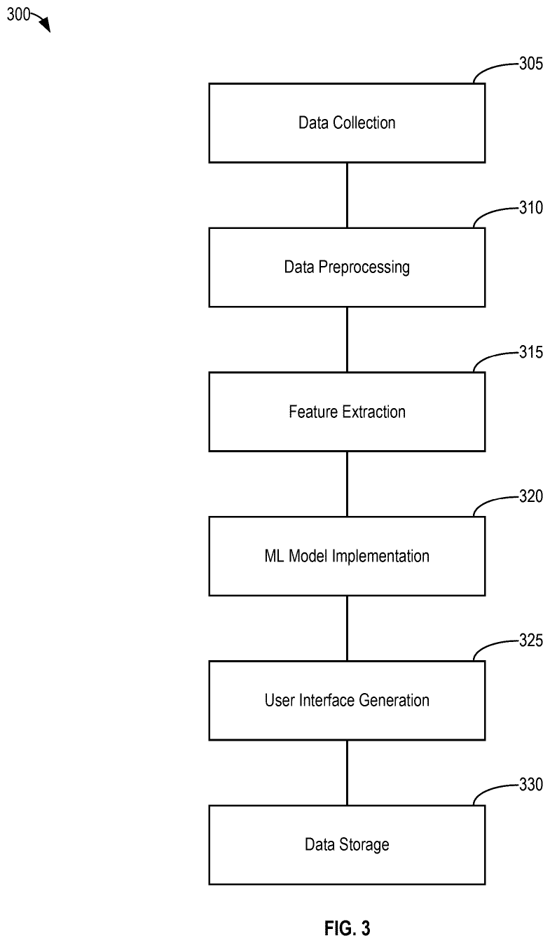

FIG. 3 depicts a flow diagram of a process 300 to monitor medical statuses, according to some embodiments. In some embodiments, at least one of the computing devices, circuits, circuitry, hardware, and/or devices described herein may perform at least one step of the process 300. For example, the processing circuit 110 may perform at least one step of the process 300. In some embodiments, at least one step of the process 300 may be repeated, reproduced, reperformed, and/or subsequently executed again.

In some embodiments, step 305 may include collecting data. For example, the sensors 157 may collect information associated with a user wearing the device 150. In some embodiments, step 305 may include collecting at least one of the various types of medical data described herein. For example, the sensors 157 may collect heart rate information.

In some embodiments, the processing circuits 110 may store, in memory 120, instructions (e.g., firmware, software, program code, executable code, etc.) to interface with and/or communicate with the sensors 157. For example, the processing circuit 110 may store instructions that cause the processors 115 to interface with the sensors 157. In some embodiments, step 305 may include a continuous and/or semi-continuous data stream. For example, the sensors 157 may continuously and/or semi-continuously provide medical data to the processing circuits 110.

In some embodiments, step 310 may include processing the data. For example, the data received in step 305 may be preprocessed. In some embodiments, the processing circuits 110 may apply one or more algorithms to filter the data received in step 305. For example, the processing circuits 110 may apply an algorithm to remove noise from the data collected by the sensors 157. As another example, the processing circuits 110 may apply an algorithm to standardize and/or modify the data collected by the sensors 157.

In some embodiments, step 315 may include extracting features. For example, the data preprocessed in step 310 may undergo feature extraction. In some embodiments, the processing circuit 110 may extract, from the data preprocessed in step 310, at least one of an average heart rate, heart rate variability, gait analysis, step counting, fall detection, identification of pressure points, balance, weight distribution, and/or interpretation of GSR data.

In some embodiments, step 320 may include implementing one or more ML models. For example, the processing circuit 110 may implement the ML model 140. In some embodiments, the processing circuit 110 may implement the ML model 140 to generate one or more predictions. For example, the processing circuit 110 may input, into the ML model 140, heart rate data, sweat levels, and/or motion data to generate a prediction of stress (e.g., stress detection). As another example, the processing circuit 110 may input data into the ML model 140 to identify movement patterns that deviate from a predetermined metric and/or value. Stated otherwise, the processing circuit 110 may implement or prompt the ML model 140 to detect deviations or changes to a gait pattern or movement pattern of the user. As even another example, the processing circuit 110 may input data into the ML model 140 to predict at least one of fall risks and/or health risks.

In some embodiments, step 325 may include generating one or more user interfaces. For example, the processing circuit 110 may transmit one or more signals to cause the user device 165 to display one or more user interfaces. In some embodiments, the user interfaces may include a dashboard to display data measured by the sensors 157. The dashboard may display medical data in one or more formats. For example, the dashboard may display medical data in a format that is interpretable by one or more caregivers. In some embodiments, the user interfaces may include one or more alerts to notify one or more caregivers.

In some embodiments, step 330 may include storing data. For example, the processing circuit 110 may transmit one or more signals to cause the remote database 135 to store medical data collected by the sensors 157. As another example, the processing circuit 110 may store medical data in memory 120. In some embodiments, the medical data may be stored to generate and/or create historical data for long-term analysis and/or to determine one or more trends. In some embodiments, the data may be stored in a manner to comply and/or satisfy one or more regulations. For example, the processing circuit 110 may store, in memory 120, the medical data in conformance with medical data regulations.

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

It should be understood that no claim element herein is to be construed under the provisions of 35 U.S.C. § 112(f), unless the element is expressly recited using the phrase “means for.”

As used herein, the term “medical record” may include information relating to the care of one or more individuals. Medical records may include physical records (e.g., paper documents, receipts, etc.) and/or electronic records (e.g., electronic medical records (“EMR”), administration records (“eMAR”), electronic treatment authorization request records (“eTAR”), activities of daily living records (“ADL”), etc.). Medical records may include informal records (e.g., physician recommendations, etc.) and/or formal records (e.g., post-operative reports, prescriptions, etc.).

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (“IC”), discrete circuits, system on a chip (“SOC”) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so

on.

The “circuit” may also include one or more processors communicatively coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (“ASICs”), field programmable gate arrays (“FPGAs”), digital signal processors (“DSPs”), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud-based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

An exemplary system for implementing the overall system or portions of the embodiments might include a computing system in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), a distributed ledger (e.g., a blockchain), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data that cause a general purpose computer, a special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

It should be understood that a “network interface,” as used herein, includes any of a cellular transceiver (e.g., Code Division Multiple Access (“CDMA”), Global System for Mobile Communications (“GSM”), Long-Term Evolution (“LTE”), etc.), a wireless network transceiver (e.g., 802.11X, ZigBee, or Bluetooth), an external network device (e.g., computer port, network interface card (“NIC”), network socket, port), or a combination thereof (e.g., both a cellular transceiver and a Bluetooth transceiver). In some arrangements, a network interface includes hardware and machine-readable media sufficient to support communication over multiple channels of data communication. Further, in some arrangements, the network interface includes cryptography capabilities to establish a secure, or relatively secure, communication session between one or more computing devices. In this regard, personal information about clients, medical records, financial data, and other types of data is encrypted and transmitted to prevent, or substantially prevent, the threat of hacking.

It should also be noted that the term “input devices” or “input components,” as described herein, may include any type of input device including, but not limited to, a keyboard, a keypad, a mouse, joystick, or other input devices performing a similar function. Comparatively, the term “output device” or “output component,” as described herein, may include any type of output device including, but not limited to, a computer monitor, printer, facsimile machine, or other output devices performing a similar function.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes, and omissions may be made in the design, operating conditions and arrangement of the embodiments without departing from the scope of the present disclosure as expressed in the appended claims.

What is claimed is:

1. A system, comprising:
a device configured to dispose within a wearable article, the device comprising:
one or more sensors configured to collect information associated with a user of the device;
a communication interface configured to communicate with one or more computing devices of the system and the one or more computing devices remote from the device;
a power source configured to power the one or more sensors, the communication interface, and one or more processing circuits of the device; and
the one or more processing circuits in communication with the one or more sensors and the communication interface, the one or more processing circuits configured to:
receive, from the one or more sensors, the information associated with the user of the device, the information including medical data of the user and the medical data measured by the one or more sensors; and
transmit, via the communication interface, responsive to receipt of the medical data, one or more first data packets to provide the medical data to the one or more computing devices; and
the one or more computing devices configured to:
receive, from the communication interface, the one or more first data packets;
retrieve, from at least one database, a data structure associated with the user;
update, responsive to retrieval of the data structure, the data structure to reflect at least a portion of the medical data; and
transmit, to the communication interface, one or more second data packets to indicate receipt of the medical data.

2. The system of claim 1, wherein the medical data includes an indication of a gait pattern of the user, and comprising:
the one or more computing devices configured to:
transmit at least one prompt to a machine learning model, the machine learning model configured to (i) receive at least a portion of the data structure, (ii) detect one or more differences between the gait pattern and at least one previously identified gait pattern of the user, and (iii) generate at least one output;
receive the at least one output from the machine learning model;
detect that the one or more differences exceed a predetermined threshold; and
provide, to a display device, an input to cause the display device to present a graphical user interface to output an indication of the one or more differences.

3. The system of claim 2, the at least one previously identified gait pattern of the user measured by the one or more sensors, the at least one previously identified gait pattern reflected in the data structure as a data array, and wherein detection of the one or more differences includes the machine learning model to:
extract, from the medical data, one or more values that pertain to the gait pattern; and
compare one or more entries of the data array with the one or more values extracted from the medical data.

4. The system of claim 1, comprising:
the one or more computing devices configured to:
implement, responsive to receipt of the one or more first data packets, a preprocessing pipeline on the medical data;
implement, responsive to completion of the preprocessing pipeline, a feature extraction pipeline on the medical data;
determine, responsive to implementation of the feature extraction pipeline, that the medical data indicates a movement pattern of the user;
provide, as at least one input, the movement pattern to a machine learning model to cause the machine learning model to generate at least one output; and
update, responsive to generation of the at least one output, the data structure to include the at least one output.

5. The system of claim 1, comprising:
the one or more computing devices configured to:
detect, responsive to retrieval of the data structure, a difference between one or more first portions of the medical data and one or more second portions of previous medical data measured by the one or more sensors;
input, responsive to detection of the difference, at least the one or more first portions of the medical data into a machine learning model trained to predict medical states;
generate, using the machine learning model, a prediction of a medical state of the user, the prediction based on an output of the machine learning model; and
transmit, responsive to generation of the prediction, one or more signals to cause a display device to display a user interface to provide an indication of the prediction of the medical state.

6. The system of claim 5, comprising:
the one or more computing devices configured to:
detect, based on the prediction of the medical state of the user, a change in a condition of the user; and
transmit, responsive to detection of the change in the condition, one or more second signals to cause the display device to produce an output to indicate the change in the condition of the user, the output including at least one of:
an audible output;
a visual output; or
a haptic output.

7. The system of claim 1, wherein the medical data measured by the one or more sensors includes at least one of:
a heart rate of the user;
an amount of perspiration of the user;
a motion level of the user;
a body temperature of the user; or
an amount of pressure applied to an area of the user.

8. The system of claim 1, wherein the one or more sensors include at least one of:
a motion sensor;
a gyroscope;
an optical heart rate sensor;
a pressure sensor;
a galvanic skin response sensor; or
a flex sensor.

9. The system of claim 1, wherein the wearable article is at least one sock, and comprising:
the one or more sensors configured to be disposed within an interior portion of the at least one sock; and
the at least one sock including at least one slot to receive the one or more sensors.

10. A distributed computing system, comprising:
a device to dispose within a wearable article, including:
one or more processing circuits configured to:
receive, from one or more sensors of the device, information associated with a user of the device, the information including medical data of the user and the medical data measured by the one or more sensors; and
transmit, via a communication interface, responsive to receipt of the medical data, one or more first data packets to provide the medical data to one or more computing devices of the distributed computing system; and
the one or more computing devices configured to:
receive, from the communication interface, the one or more first data packets;
retrieve, from at least one database, a data structure associated with the user;
update, responsive to retrieval of the data structure, the data structure to reflect at least a portion of the medical data; and
transmit, to the communication interface, one or more second data packets to indicate receipt of the medical data measured by the one or more sensors.

11. The distributed computing system of claim 10, wherein the medical data includes an indication of a gait pattern of the user, and comprising:
the one or more computing devices configured to:
transmit at least one prompt to a machine learning model, the machine learning model configured to (i) receive at least a portion of the data structure, (ii) detect one or more differences between the gait pattern and at least one previously identified gait pattern of the user, and (iii) generate at least one output;
receive the at least one output from the machine learning model;
detect that the one or more differences exceed a predetermined threshold; and
provide, to a display device, an input to cause the display device to present a graphical user interface to output an indication of the one or more differences.

12. The distributed computing system of claim 11, the at least one previously identified gait pattern of the user measured by the one or more sensors, the at least one previously identified gait pattern reflected in the data structure as a data array, and wherein detection of the one or more differences includes the machine learning model to:
extract, from the medical data, one or more values that pertain to the gait pattern; and
compare one or more entries of the data array with the one or more values extracted from the medical data.

13. The distributed computing system of claim 10, comprising:
the one or more computing devices configured to:
implement, responsive to receipt of the one or more first data packets, a preprocessing pipeline on the medical data;
implement, responsive to completion of the preprocessing pipeline, a feature extraction pipeline on the medical data;
determine, responsive to implementation of the feature extraction pipeline, that the medical data indicates a movement pattern of the user;
provide, as at least one input, the movement pattern to a machine learning model to cause the machine learning model to generate at least one output; and
update, responsive to generation of the at least one output, the data structure to include the at least one output.

14. The distributed computing system of claim 10, comprising:
the one or more computing devices configured to:
detect, responsive to retrieval of the data structure, a difference between one or more first portions of the medical data and one or more second portions of previous medical data measured by the one or more sensors;
input, responsive to detection of the difference, at least the one or more first portions of the medical data into a machine learning model trained to predict medical states;
generate, using the machine learning model, a prediction of a medical state of the user, the prediction based on an output of the machine learning model; and
transmit, responsive to generation of the prediction, one or more signals to cause a display device to display a user interface to provide an indication of the prediction of the medical state.

15. The distributed computing system of claim 14, comprising:
the one or more computing devices configured to:
detect, based on the prediction of the medical state of the user, a change in a condition of the user; and
transmit, responsive to detection of the change in the condition, one or more second signals to cause the display device to produce an output to indicate the change in the condition of the user, the output including at least one of:
an audible output;
a visual output; or
a haptic output.

16. A device to dispose within a wearable article, the device comprising:
one or more sensors configured to collect information associated with a user of the device;
a communication interface configured to communicate with one or more computing devices and the one or more computing devices remote from the device;
a power source configured to power the one or more sensors, the communication interface, and one or more processing circuits of the device; and
the one or more processing circuits in communication with the one or more sensors and the communication interface, the one or more processing circuits configured to:
receive, from the one or more sensors, the information associated with the user of the device, the information including medical data of the user and measured by the one or more sensors;
transmit, via the communication interface, responsive to receipt of the medical data, one or more first data packets to provide the medical data to the one or more computing devices; and
receive, from the one or more computing devices, one or more second data packets to indicate an update to a data structure associated with the user.

17. The device of claim 16, wherein the medical data includes an indication of a gait pattern of the user, and wherein the one or more computing devices are configured to:
transmit at least one prompt to a machine learning model, the machine learning model configured to (i) receive at least a portion of the data structure, (ii) detect one or more differences between the gait pattern and at least one previously identified gait pattern of the user, and (iii) generate at least one output;
receive the at least one output from the machine learning model;
detect that the one or more differences exceed a predetermined threshold; and
provide, to a display device, an input to cause the display device to present a graphical user interface to output an indication of the one or more differences.

18. The device of claim 16, wherein the medical data measured by the one or more sensors includes at least one of:
a heart rate of the user;
an amount of perspiration of the user;
a motion level of the user;
a body temperature of the user; or
an amount of pressure applied to an area of the user.

19. The device of claim 16, wherein the one or more sensors include at least one of:
a motion sensor;
a gyroscope;
an optical heart rate sensor;
a pressure sensor;
a galvanic skin response sensor; or
a flex sensor.

20. The device of claim 16, wherein the wearable article is at least one sock, and comprising:
the one or more sensors configured to be disposed within an interior portion of the at least one sock; and
the at least one sock including at least one slot to receive the one or more sensors.
